(19) United States
(12) Patent Application Publication
(10) Pub. No.: US 2022/0409763 A1
(43) Pub. Date: Dec. 29, 2022

(54) DEVICE FOR DIFFUSING VOLATILE SUBSTANCES

(71) Applicant: Zobele Holding, S.P.A., Trento (IT)

(72) Inventors: Stefano Deflorian, Trento (IT); Walter Sordo, Trento (IT); Ruben Garcia, Barcelona (ES)

(21) Appl. No.: 17/756,181

(22) PCT Filed: Nov. 18, 2020
(86) PCT No.: PCT/EP2020/082525

§ 371 Date: May 18, 2022

(30) Foreign Application Priority Data
Nov. 19, 2019 (EP) 19210007.1

(51) Int. Cl. A61L 9/12; A01N 25/18; A01P 7/04; A01M 1/20

(52) U.S. Cl. CPC A61L 9/12; A01N 25/18; A01P 7/04; A01M 1/2055

(57) ABSTRACT

The device for diffusing volatile substances, comprises a container (1) that contains a volatile substance (2); a perforable sheet (3) that closes at least a portion of said container (1); at least one perforating element (4) that can perforate said perforable sheet (3); and a porous element (5) that is impregnated with the volatile substance (2) when the perforable sheet (3) is perforated; wherein the porous element (5) comprises at least one weakened area (6) in correspondence with said at least one perforating element (4).

It permits to provide a device for diffusing volatile substances where an accidental leakage is avoided.

The present invention refers to a device for diffusing volatile substances.

### BACKGROUND OF THE INVENTION

Among volatile substance diffusers, one of the simplest products existing, and yet still on the top of sales, is impregnated cellulosic support to be hanged for example in a car or in a wardrobe.

Apart the simplest embodiment of cellulose impregnated at the factory, some improvements have been proposed where a container containing the liquid is perforated to release the liquid in a porous element from which it is then released in the atmosphere.

Examples of this kind of volatile substance diffusers are disclosed in U.S. Pat. Nos. 4,526,320, 4,762,275, 4,161,284, DE2807424, GB2228681, and GB2349089.

Most of these diffusers comprise an activation part including a wide sharp tip or needle that is placed in proximity of a breakable portion of the container.

From these documents, it is clear from a skilled person that it is very challenging to have such a sharp tip in contact with a breakable foil and not to have an accidental activation of the system, either during the assembly process or during transportation.

Another drawback of the current diffusers is that the needle or sharp tip is big, forming a big hole in the breakable portion of the container, that could create non-wanted alteration of the aesthetics of the refill, especially if this part is used for containing the brand of the product.

### DESCRIPTION OF THE INVENTION

Therefore, one purpose of the present invention is to provide a device for diffusing volatile substances where an accidental leakage is avoided.

With the device for diffusing volatile substances according to the present invention it is possible to solve said drawbacks, providing other advantages that are described below.

The device for diffusing volatile substances according to the present invention comprises:
a container that contains a volatile substance;
a perforable sheet that closes at least a portion of said container;
at least one perforating element that can perforate said perforable sheet; and
a porous element that is impregnated with the volatile substance when the perforable sheet is perforated;

wherein the porous element comprises at least one weakened area in correspondence with said at least one perforating element.

Furthermore, said at least one perforating element is preferably mounted on a plate, which when the perforable sheet is perforated, is in contact with the porous element.

According to a preferred embodiment, the weakened area is a cut or several cuts made in said porous element, e.g. the weakened are is formed by two crossing cuts defining a central point.

Advantageously, said plate includes two or more perforating elements, wherein the perforating elements in said plate are placed vertically, defining an upper perforating element and a lower perforating element.

According to this embodiment, the container is partially filled with the volatile substance, defining a filled portion and an empty portion in the container so that the upper perforating element is at a height corresponding to the empty portion and the lower perforating element is at a height corresponding to the filled portion.

To prevent the removal of the perforating element(s), the or each perforating element comprises a stopper at its tip. The stopper prevents the activation system to go back and the top side can show that the perforation has happen and it does not have to be repeated by the user.

At the same time, if the distance between the stopper and the top part is the same thickness of the porous element, the top part will remain in contact with the porous element and will prevent the likelihood of liquid to freely come out from the hole created between the perforable sheet and the external surface of porous element.

With the device according to the present invention, the perforation of the perforable sheet can be made easily, and once perforated, an accidental leakage is prevented.

### BRIEF DESCRIPTION OF THE DRAWINGS

For a better understanding the above explanation and for the sole purpose of providing an example, some non-limiting drawings are included that schematically depict a practical embodiment.

FIG. 1 is a perspective of a portion of the device for diffusing volatile substances according to the present invention;

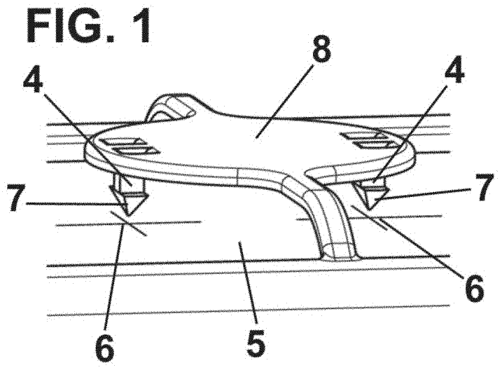

FIG. 2 is a sectional view of a portion of the device for diffusing volatile substances according to the present invention before its activation; and

FIG. 3 is a sectional view of a portion of the device for diffusing volatile substances according to the present invention after its activation, when the perforation element has perforated the perforable sheet.

### DESCRIPTION OF A PREFERRED EMBODIMENT

The present invention refers to a device for diffusing volatile substances, such as perfume or insecticide, comprising a container 1 containing a liquid volatile substance 2, having at least on part of it that is easily perforable sheet 3.

It must be pointed out that the device according to the present invention can comprise more that one container, but for simplicity reasons only one container is shown in the drawings.

The device for diffusing volatile substances according to the present invention also comprises one or more perforating elements 4, such as needles, which are designed to be able to perforate the perforable sheet 3 of the container 1 when user press them against the perforable sheet 3.

The device for diffusing volatile substances according to the present invention also comprises a porous element 5 that is able to absorb and contain the liquid after the perforable sheet 3 of the container 1 has been perforated.

This porous element 5 is placed between the perforating elements 4 and the perforable sheet 3, and the porous element 5 comprises a weakened area 6 in the proximity of the perforating elements 4.

Furthermore, the perforating elements 4 have a design that avoid that it goes back after perforating the perforable sheet 3 of the container 1. To this end, e.g. each perforating element 4 has a stopper 7 or a portion with greater dimensions than the rest of the perforating element 4, e.g. an arrow shape, i.e. the cross section of the perforation element 4 is progressively wider distally from its tip.

This way, after perforating the perforable sheet 3, the perforating element 4 cannot be removed from the container 1, avoiding an accidental leakage.

According to the embodiment shown in the drawing, two perforating elements 4 are mounted on a plate 8 that covers the external part of the weakened area of the porous element 5.

As the perforating elements 4 are not able to be removed from the container 1, the plate 8 is maintained in physical contact with the porous element 5 after activation, avoiding liquid to flow freely through the weakened area 6 of the porous element 5.

Preferably, the container 1 is made from a plastic cup, thermoformed, closed by a rupturing foil.

The rupturing foil is preferably a very thin aluminium foil with a sealing layer on one side and a protective lacquer on the other.

The porous element 5 is preferably a cellulosic support. Alternatively, it could be made of plastic fibers or plastic foam.

The weakened area 6, according to a preferred embodiment, is a cut or a partial cut with a cross shape, where the center of the cross matches with the tip of the perforating element 4.

Alternatively, the weakened area 6 can be made by a hole in the porous element 5.

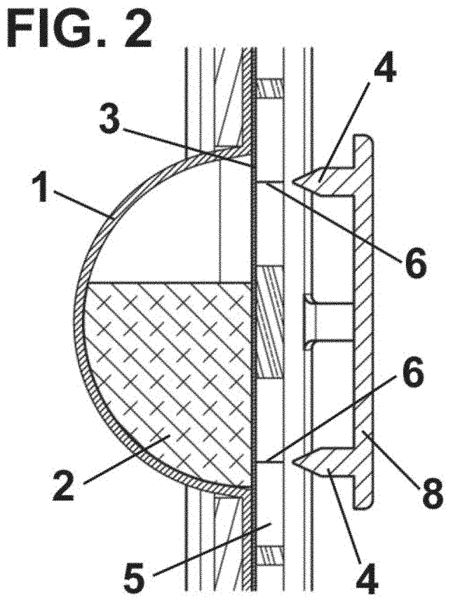

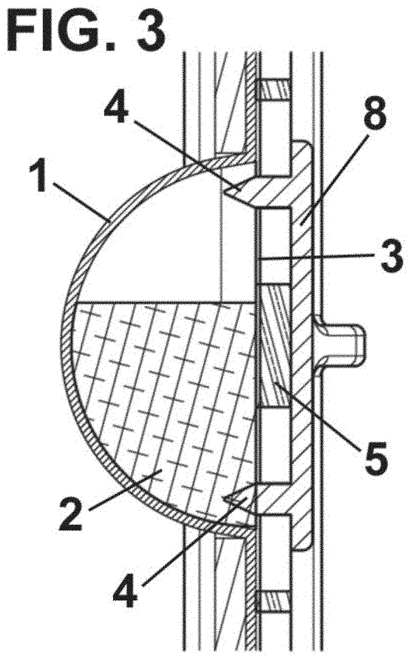

According to an embodiment, shown in FIGS. 2 and 3, the device according to the present invention comprises two perforating elements 4, placed vertically to each other, where the upper perforating element 4 is at a height where, inside the container 1, there is no liquid, and the lower perforating element 4 is at a height corresponding to the bottom of the container 1. This way, the user can form a hole in the perforable sheet 3 yet still having the liquid to flow through it (the upper hole aim is to allow air to enter in the container 1).

In a preferred embodiment, the device according to the present invention is a device to be hanged to a car mirror with an overall dimension of e.g. 13×8 cm.

The device can comprise a U-shape plastic frame, preferably made of polypropylene, where four containers 1 can be made, containing the liquid volatile substance 2.

The perimetric wall of the plastic frame is done with a U-shape profile in order to guide and retain the porous element 5 in position.

In the back part of the frame, four plates 8 with activation perforating elements 4 can be formed, each plate for one container. These plates 8 can be individually pressed by a user to force the perforating elements 4 to perforate the perforable sheet 3.

The four containers 1 are preferably filled with the same amount and kind of fragrance. E.g. each container contains the volume of fragrance equivalent of a duration of 1 week.

For using the device according to the present invention, the user just activates it pressing on the plate 8 so that the perforating element(s) 4 perforate(s) the perforable 3, and the porous element 5 is impregnated with the liquid volatile substance 2.

Even though reference has been made to a specific embodiment of the invention, it is obvious for a person skilled in the art that the device for diffusing volatile substances described herein is susceptible to numerous variations and modifications, and that all of the details mentioned can be substituted for other technically equivalent ones without departing from the scope of protection defined by the attached claims.

1. A device for diffusing volatile substances, comprising:
a container that contains a volatile substance;
a perforable sheet that closes at least a portion of said container;
at least one perforating element that can perforate said perforable sheet; and
a porous element that is impregnated with the volatile substance when the perforable sheet is perforated;
wherein the porous element comprises at least one weakened area in correspondence with said at least one perforating element.

2. The device for diffusing volatile substances according to claim 1, wherein said at least one perforating element is mounted on a plate, which when the perforable sheet is perforated, is in contact with the porous element.

3. The device for diffusing volatile substances according to claim 1, wherein the weakened area is a cut or several cuts made in said porous element.

4. The device for diffusing volatile substances according to claim 3, wherein the weakened area is formed by two crossing cuts defining a central point.

5. The device for diffusing volatile substances according to claim 2, wherein said plate includes two or more perforating elements.

6. The device for diffusing volatile substances according to claim 1, wherein the or each perforating element comprises a stopper at its tip.

7. The device for diffusing volatile substances according to claim 5, wherein the perforating elements in said plate are placed vertically, defining an upper perforating element and a lower perforating element.

8. The device for diffusing volatile substances according to claim 7, wherein the container is partially filled with the volatile substance, defining a filled portion and an empty portion in the container so that the upper perforating element is at a height corresponding to the empty portion and the lower perforating element is at a height corresponding to the filled portion.
